(19) United States
(12) Patent Application Publication
(10) Pub. No.: US 2025/0327766 A1
(43) Pub. Date: Oct. 23, 2025

(54) GAS SENSOR AND GAS SENSING METHOD USING THE SAME

(71) Applicant: NATIONAL TAIWAN UNIVERSITY, Taipei City (TW)

(72) Inventors: Yi-Chia CHOU, Taipei City (TW); Chang-Hsun HUANG, Taipei City (TW); Wen-Chien MIAO, Taipei City (TW)

(21) Appl. No.: 18/773,954

(22) Filed: Jul. 16, 2024

(30) Foreign Application Priority Data
Apr. 19, 2024 (TW) 113114606

(51) Int. Cl. G01N 27/12; G01N 33/00

(52) U.S. Cl. CPC G01N 27/127; G01N 33/0027

(57) ABSTRACT

The present invention relates to a gas sensor, comprising a substrate, a first electrode and a second electrode. The substrate is provided with a gas sensing layer, the gas sensing layer comprises a gallium nitride nanowire and a plurality of silicon nanowires, and the plurality of silicon nanowires are grown on the gallium nitride nanowire. The first electrode is disposed on the substrate, and the second electrode is connected to the gas sensing layer. Moreover, the present invention relates to a gas sensing method using the aforementioned gas sensor.

### CROSS-REFERENCE TO RELATED APPLICATIONS

This application claims the benefits of the Taiwan Patent Application Serial Number 113114606, filed on Apr. 19, 2024, the subject matter of which is incorporated herein by reference.

### BACKGROUND OF THE INVENTION

### Field

The present invention relates to a gas sensor and a gas sensing method using the same. In particular, the present invention relates to a gas sensor with high sensing efficiency at room temperature and a gas sensing method using the same.

### Description of Related Art

There are many types of gas sensors, and their working principles are also different. They have been widely used in fields such as semiconductor manufacturing, medical diagnosis, environmental monitoring, or national security. However, conventional gas sensors have higher requirements for gas sensing conditions. Taking the ammonia sensor as an example, its minimum gas sensing concentration needs to be greater than 1 ppm. While reducing the minimum gas sensing concentration, more complex process steps or higher operating temperatures are required, thus making the cost high.

In addition, the conventional gas sensor has poor selectivity for gases,

and when multiple gases are present at the same time, they easily interfere with each other and affect the sensing results. It can be seen that the conventional gas sensor has one or more of the following problems: high manufacturing cost, poor gas selectivity, low gas sensitivity, and high operating temperature, which makes the usage conditions of the gas sensor quite limited.

For this reason, the inventor, in the spirit of active invention, is desirable to propose a novel gas sensor and a gas sensing method using the same, especially without complicated process steps or higher operating temperatures, or it can effectively reduce the minimum gas sensing concentration and has better gas selectivity and sensitivity to eliminate or alleviate the above problems.

### SUMMARY OF THE INVENTION

One object of the present invention is to provide a gas sensor and a gas sensing method using the same. Gas sensing is performed using a gas sensing layer comprising a GaN nanowire and a plurality of silicon nanowires grown thereon. This effectively improves the gas sensing efficiency, can reach the lowest gas sensing concentration of ppb level at room temperature, and has excellent selectivity for gases, which can reduce interference from other gases; or the gas sensor can be easily prepared without complicated manufacturing steps.

In view of this, according to one aspect of the present invention, a gas sensor is provided, which comprises a substrate, a first electrode and a second electrode. The substrate is provided with a gas sensing layer, the gas sensing layer comprises a GaN nanowire and a plurality of silicon nanowires grown on the GaN nanowire. The first electrode is disposed on the substrate, and the second electrode is connected to the gas sensing layer.

In the present invention, the diameter of the GaN nanowire may range from 50 nm to 500 nm, for example, from 100 nm to 500 nm, from 120 nm to 500 nm, from 150 nm to 500 nm, from 150 nm to 400 nm, from 200 nm to 400 nm, from 200 nm to 300 nm, or about 250 nm; but the present invention is not limited thereto.

In the present invention, the length of the GaN nanowire may range from 0.5 μm to 10 μm, for example, from 0.5 μm to 8 μm, from 0.5 μm to 5 μm, from 0.5 μm to 3 μm, from 0.8 μm to 8 μm, from 0.8 μm to 5 μm, from 0.8 μm to 3 μm, from 1 μm to 8 μm, from 1 μm to 5 μm, from 1 μm to 3 μm, or about 1.5 μm; but the present invention is not limited thereto.

In the present invention, the diameters of the plurality of silicon nanowires may respectively range from 10 nm to 300 nm, for example, from 10 nm to 200 nm, from 10 nm to 150 nm, from 10 nm to 100 nm, from 10 nm to 50 nm, from 15 nm to 100 nm, from 30 nm to 80 nm, or about 50 nm; but the present invention is not limited thereto.

In the present invention, the lengths of the plurality of silicon

nanowires may respectively range from 10 nm to 500 nm, for example, from 10 nm to 400 nm, 10 nm to 300 nm, 10 nm to 200 nm, 50 nm to 400 nm, 50 nm to 300 nm, 50 nm to 200 nm, or about 100 nm; but the present invention is not limited thereto.

In the present invention, one end of the plurality of silicon nanowires away from the GaN nanowire may be respectively provided with a catalyst particle. The material of the catalyst particle may be selected from the group consisting of Au, Ag, Ni and an alloy thereof. For example, the material of the catalyst particle may be Au, Ag, Ni or Ag-Au alloy; but the present invention is not limited thereto.

In the present invention, the first electrode may be silver glue, and the second electrode may be a grid electrode; but the present invention is not limited thereto.

In the present invention, the gas sensor may be used to sense ammonia; but the present invention is not limited thereto.

In the present invention, the GaN nanowire and the plurality of silicon nanowires may form a branching structure; but the present invention is not limited thereto.

According to another aspect of the present invention, a gas sensing method is provided, which comprises the following steps: providing the aforesaid gas sensor; placing the gas sensor in a container; introducing a gas to be measured at a predetermined flow rate for a period of time, and measuring a current change difference of the gas sensor; and converting the current change difference to obtain a concentration of the gas to be measured.

In the present invention, the predetermined flow rate may range from 100 mL/min to 1000 mL/min, for example, from 200 mL/min to 1000 mL/min, from 200 mL/min to 900 mL/min, from 200 mL/min to 800 mL/min, from 300 mL/min to 900 mL/min, from 300 mL/min to 800 mL/min, from 300 mL/min to 700 mL/min, from 300 mL/min to 600 mL/min, from 400 mL/min to 600 mL/min, or about 500 mL/min; but the present invention is not limited thereto.

In the present invention, the time for introducing the gas may range from 1 second to 60 seconds, for example, from 3 seconds to 60 seconds, from 5 seconds to 60 seconds, from 10 seconds to 60 seconds, from 30 seconds to 60 seconds, from 1 second to 45 seconds, from 1 second to 30 seconds, about 1 second, about 5 seconds, about 10 seconds, about 30 seconds, or about 60; but the present invention is not limited thereto.

In the present invention, the gas to be measured may be ammonia; but the present invention is not limited thereto.

Other novel objects, advantages and features of the present invention will become more apparent from the following detailed description when taken in conjunction with the accompanying drawings.

### BRIEF DESCRIPTION OF DRAWINGS

FIG. 1 is a schematic diagram of a gas sensor according to Embodiment 1 of the present invention.

FIG. 2 is a schematic diagram showing the process for manufacturing a gas sensor according to Embodiment 1 of the present invention.

FIG. 3 is a schematic diagram of a measuring device comprising a gas sensor according to Embodiment 1 of the present invention.

FIG. 4 to FIG. 8 respectively show the electrical diagrams of Embodiment 1 to Embodiment 5 at different ammonia concentrations.

FIG. 9 shows the relationship between different ammonia concentrations and responses from Embodiment 1 to Embodiment 5.

FIG. 10 is a partial enlarged view of the dotted line in FIG. 9.

FIG. 11 shows the electrical diagram of Comparative embodiment 1.

### DETAILED DESCRIPTION OF THE INVENTION

Different embodiments of the present invention are provided in the following description. These embodiments are meant to explain the technical content of the present invention, but not meant to limit the scope of the present invention. A feature described in an embodiment may be applied to other embodiments by suitable modification, substitution, combination, or separation.

It should be noted that, in the present specification, when a component is described to have an element, it means that the component may have one or more of the elements, and it does not mean that the component has only one of the element, except otherwise specified.

Moreover, in the present specification, the ordinal numbers, such as “first” or “second”, are used to distinguish a plurality of elements having the same name, and it does not mean that there is essentially a level, a rank, an executing order, or a manufacturing order among the elements, except otherwise specified. A “first” element and a “second” element may exist together in the same component, or alternatively, they may exist in different components, respectively. The existence of an element described by a greater ordinal number does not essentially mean the existent of another element described by a smaller ordinal number.

In the present specification, unless otherwise specified, the so-called A “or” or “and/or” B means that A exists alone, B exists alone, or A and B exists simultaneously; the so-called A “and” B means that A and B exist at the same time; and the so-called “includes”, “comprises”, “has”, and “contains” ”means including but not limited to this.

Moreover, in the present specification, the terms, such as “top” “bottom”, “left”, “right”, “front”, “back”, or “middle”, as well as the terms, such as “on”, “above”, “under”, “below”, or “between”, are used to describe the relative positions among a plurality of elements, and the described relative positions may be interpreted to include their translation, rotation, or reflection.

Moreover, in the present specification, when an element is described to be arranged “on” another element, it does not essentially mean that the elements contact the other element, except otherwise specified. Such interpretation is applied to other cases similar to the case of “on”.

Moreover, in the present specification, a value may be interpreted to cover a range within ±10% of the value, and in particular, a range within ±5% of the value, except otherwise specified; a range may be interpreted to be composed of a plurality of subranges defined by a smaller endpoint, a smaller quartile, a median, a greater quartile, and a greater endpoint, except otherwise specified.

### Embodiment 1

### Structure of Gas Sensor

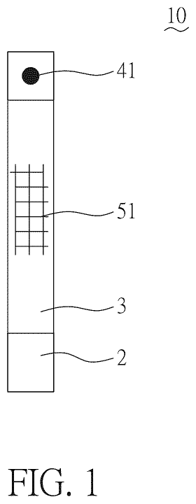

FIG. 1 is a schematic diagram of a gas sensor according to Embodiment 1 of the present invention.

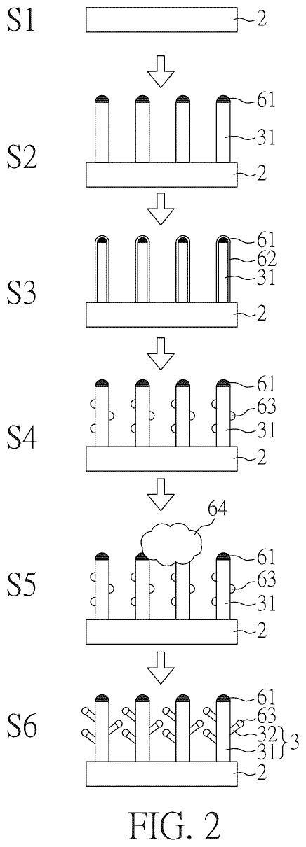

As shown in FIG. 1, the gas sensor 10 of the present embodiment comprises: a substrate 2, a gas sensing layer 3, a first electrode 41 and a second electrode 51. The substrate 2 is provided with the gas sensing layer 3, the gas sensing layer 3 comprises GaN nanowires 31 and silicon nanowires 32 (as shown in FIG. 2) grown on the GaN nanowires 31 (as shown in FIG. 2). In addition, the first electrode 41 is disposed on the substrate 2, and the second electrode 51 is connected to the gas sensing layer 3.

In the present embodiment, the substrate 2 is a P-type heavy doped silicon (111) substrate. The diameter of the GaN nanowire 31 is about 250 nm and the length thereof is about 1.5 μm. The diameter of the silicon nanowire 32 is about 50 nm and the length thereof is about 100 nm. The first electrode 41 is silver glue, and the second electrode 51 is a grid Ti-Au electrode. However, the present invention is not limited thereto.

### Method for Manufacturing Gas Sensor

FIG. 2 is a schematic diagram showing the process for manufacturing a gas sensor according to Embodiment 1 of the present invention.

### Step S1

As shown in FIG. 1 and FIG. 2, a substrate 2 was provided and cleaned by using acetone, isopropyl alcohol, and deionized water solution sequentially with ultra-sonication for 5 minutes to remove organic pollutants on the surface. The cleaned substrate 2 was immersed in a silicon dioxide etching solution (buffer oxide etch, BOE) for 10 minutes to remove the native oxide layer on the surface of the substrate 2. During the immersion process, the hydrogen atoms in the silicon dioxide etching solution will bond with the silicon atoms on the surface of the substrate 2 to form a protective layer to protect the surface of the substrate 2 from being oxidized in a short period of time. Then, a nitrogen gun was used to blow the remaining solution on the surface of the substrate 2 until it evaporated and the substrate 2 was stored in a vacuum environment. Here, the components of the silicon dioxide etching solution included amine fluoride (NH₄F) and hydrofluoric acid (HF), and their volume ratio was 7:1 (amine fluoride: hydrofluoric acid).

### Step S2

The first catalyst 61 was plated on the substrate 2 using the hydride vapor phase epitaxy (HVPE) method. The first catalyst 61 was a nickel-gold alloy, but the present invention is not limited thereto. Next, the growth temperature was controlled at about 880° C., and hydrogen chloride gas (HCl) was reacted with liquid gallium (Ga) first to generate gallium chloride (GaCl) gas, and then the gallium chloride (GaCl) gas was reacted with ammonia (NH₃) to obtain the final product, GaN nanowires 31 deposited on the substrate 2 to form a first composite material. The main reaction formulas are shown by the following Formula 1 and Formula 2.

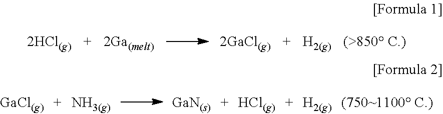

### Step S3

The thermal evaporation function of the ultra-high vacuum chemical vapor deposition (UHV-CVD) system was used, and tantalum (Ta) wire was used as a heating coil to be wound around the periphery of the crucible. Current was applied to heat the second catalyst 62 in the crucible to evaporate the second catalyst 62, a quartz crystal monitor (QCM) was used to detect the thickness of the plated second catalyst 62, and the second catalyst 62 was plated on the first composite material to form a second composite material. In the present embodiment, the second catalyst 62 was Au, but the present invention is not limited thereto.

### Step S4

Current was applied to the second composite material to perform annealing by direct heating, so that the second catalyst 62 formed into catalyst particles 63 in the form of metal droplets required for growing the silicon nanowires 32 to form a third composite material. The process temperature required for subsequent growth was maintained.

### Step S5

Precursor 64 was introduced into the ultra-high vacuum chemical vapor deposition system. The chamber base pressure of the ultra-high vacuum chemical vapor deposition system was controlled at 4×10⁻¹⁰ Torr, and the precursor 64 was 10% silane (SiH₄) diluted with argon, but the present invention is not limited thereto.

### Step S6

When the precursor 64 reached the surface of the third composite material, since the third composite material was maintained at a high temperature after being annealed, the precursor 64 was thermally decomposed. The reaction formula is shown by the following Formula 3.

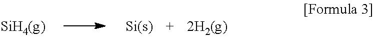

While the precursor 64 was thermally decomposed, the silicon obtained after decomposition was affected by the concentration difference and continued to dissolve into the catalyst particles 63. After silicon reached supersaturation in the catalyst particle 63, it precipitated at the interface between the catalyst particle 63 and the surface of the GaN nanowire 31 to form a plurality of silicon nanowires 32. Thus, a gas sensing layer 3 was obtained.

The obtained gas sensing layer 3 was coated with a photoresist layer by spin coating. After utilizing the grid-shaped photomask to develop, it was sent to the electron beam evaporation system to plate the second electrode material. Finally, the photoresist was removed by soaking in an acetone solution, leaving the grid-shaped second electrode 51, and the first electrode 41 was applied on the substrate 2 to obtain the gas sensor 10.

### Gas Sensing Method

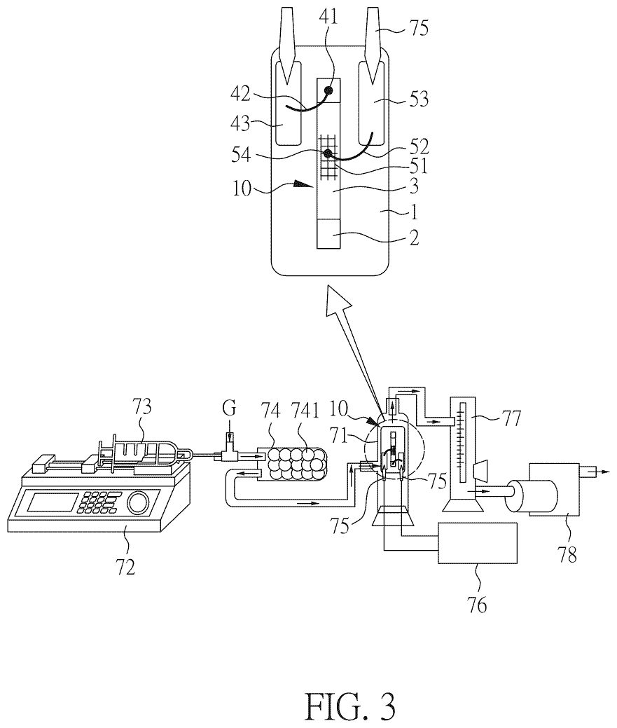

FIG. 3 is a schematic diagram of a measuring device comprising a gas sensor according to Embodiment 1 of the present invention.

The gas sensing method of the present embodiment includes the following steps.

### Step 1

As shown in FIG. 1, a gas sensor 10 of Embodiment 1 was provided.

### Step 2

As shown in FIG. 3, the gas sensor 10 disposed on the stage 1 was placed in a container 71. The first electrode 41 was electrically connected to the third electrode 43 through the first wire 42, and the second electrode 51 was electrically connected to the fourth electrode 53 through the second wire 52 and the silver glue 54. The third electrode 43 and the fourth electrode 53 were electrically connected to the U2722A modular power measurement device 76 through the alligator clips 75. The U2722A modular power supply measurement device 76 is a device for measuring electrical properties that is well known to those skilled in the art, so it will not be described in detail here. Then, the background air G was removed through the flow meter 77 and the exhaust pump 78, and the air G was introduced as a background gas at the flow rate of 500 mL/min for 15 minutes. The introduced air G first passed through the dehumidification pipe 74, and the gas humidity was controlled at 15% through the sodium hydroxide solid particles 741 in the dehumidification pipe 74. Then, the introduced air G entered into the container 71 to stabilize the measured current value.

### Step 3

An air-tight needle 73 was connected to the gas cylinder (not shown in the figure). The gas to be measured in the air-tight needle 73 was introduced by pushing the air-tight needle 73 at the advancement rate set by the stepper motor 72, so that the gas to be measured was continuously introduced for a period of time. The introduced gas to be measured was first mixed with the air G. After being processed through the dehumidification pipe 74, it entered the container 71 at a predetermined flow rate. Then, the U2722A modular power supply measurement device 76 was used to measure the current change difference of the gas sensors 10 before and after introducing the gas to be measured. The concentration of the gas to be measured in the container 71 can be calculated by the following formula 4.

$$Background gas flow rate ( mL / min ) \times Concentration of the gas to be measured ( ppm ) = Advancement rate ( mL / min ) \times Standard gas cylinder concentration ( ppm ) \qquad [ Formula 4 ]$$

*In the present embodiment, the background gas flow rate was 500 mL/min, and the standard gas cylinder concentration was 270 ppm.

### Step 4

The current change difference was converted to obtain a concentration of the gas to be measured.

In the present embodiment, the container 71 was a glass tube, the predetermined flow rate was 500 mL/min, the gas to be measured was ammonia with different concentrations (10 ppm (the advancement rate was 18.518 mL/min), 5 ppm (the advancement rate was 9.259 mL/min) or 3 ppm (the advancement rate was 5.556 mL/min)), and the introducing time was 1 second.

### Embodiment 2

The present embodiment is similar to Embodiment 1. The difference is that the gas to be measured was ammonia with different concentrations (10 ppm (the advancement rate was 18.518 mL/min), 5 ppm (the advancement rate was 9.259 mL/min), 3 ppm (the advancement rate was 5.556 mL/min), 1 ppm (the advancement rate was 1.8518 mL/min) or 0.5 ppm (the advancement rate was 0.926 mL/min)), and the introducing time was 5 seconds.

### Embodiment 3

The present embodiment is similar to Embodiment 1. The difference is that the gas to be measured was ammonia with different concentrations (10 ppm (the advancement rate was 18.518 mL/min), 5 ppm (the advancement rate was 9.259 mL/min), 3 ppm (the advancement rate was 5.556 mL/min), 1 ppm (the advancement rate was 1.8518 mL/min), 0.5 ppm (the advancement rate was 0.926 mL/min) or 0.3 ppm (the advancement rate was 0.556 mL/min)), and the introducing time was 10 seconds.

### Embodiment 4

The present embodiment is similar to Embodiment 1. The difference is that the gas to be measured was ammonia with different concentrations (10 ppm (the advancement rate was 18.518 mL/min), 5 ppm (the advancement rate was 9.259 mL/min), 3 ppm (the advancement rate was 5.556 mL/min), 1 ppm (the advancement rate was 1.8518 mL/min), 0.5 ppm (the advancement rate was 0.926 mL/min), 0.3 ppm (the advancement rate was 0.556 mL/min) or 0.1 ppm (the advancement rate was 0.1852 mL/min)), and the introducing time was 30 seconds.

### Embodiment 5

The present embodiment is similar to Embodiment 1. The difference is that the gas to be measured was ammonia with different concentrations (10 ppm (the advancement rate was 18.518 mL/min), 5 ppm (the advancement rate was 9.259 mL/min), 3 ppm (the advancement rate was 5.556 mL/min), 1 ppm (the advancement rate was 1.8518 mL/min), 0.5 ppm (the advancement rate was 0.926 mL/min), 0.3 ppm (the advancement rate was 0.556 mL/min) or 0.1 ppm (the advancement rate was 0.1852 mL/min)), and the introducing time was 60 seconds.

### Comparative Embodiment 1

Comparative embodiment 1 is similar to Embodiment 1. The difference is that, in Comparative embodiment 1, the gas to be measured was 20 ppm nitrogen dioxide and the introducing time was 20 seconds.

### Comparative embodiment 2

Comparative embodiment 2 is similar to Embodiment 1. The difference is that, in Comparative embodiment 2, the gas to be measured was 15 ppm acetone and the introducing time was 10 seconds.

### Comparative Embodiment 3

Comparative embodiment 3 is similar to Embodiment 1. The difference is that, in Comparative embodiment 3, the gas to be measured was 15 ppm acetone and the introducing time was 20 seconds.

### Comparative Embodiment 4

Comparative embodiment 4 is similar to Embodiment 1. The difference is that, in Comparative embodiment 4, the gas to be measured was 1 ppm hydrogen sulfide and the introducing time was 10 seconds.

### Comparative Embodiment 5

Comparative embodiment 5 is similar to Embodiment 1. The difference is that, in Comparative embodiment 5, the gas to be measured was 2.5 ppm carbon monoxide and the introducing time was 10 seconds.

### Comparative Embodiment 6

Comparative embodiment 6 is similar to Embodiment 1. The difference is that, in Comparative embodiment 6, the gas to be measured was 10 ppm nitric oxide and the introducing time was 10 seconds.

### Comparative Embodiment 7

Comparative embodiment 7 is similar to Embodiment 1. The difference is that, in Comparative embodiment 7, the gas to be measured was 10 ppm nitric oxide and the introducing time was 20 seconds.

### Minimum Sensing Concentration of Gas Sensor

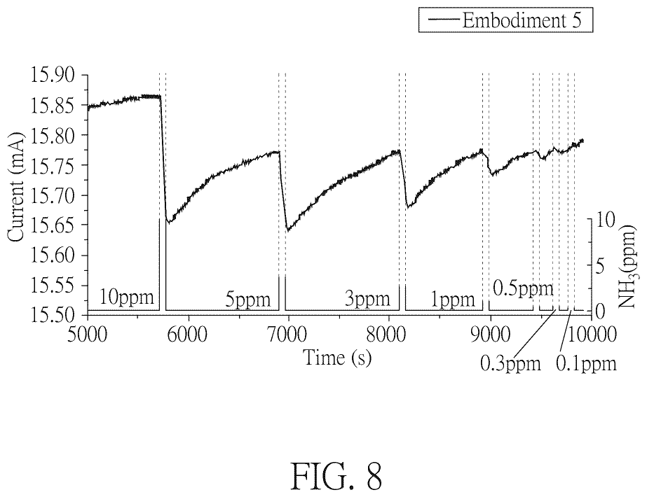

FIG. 4 to FIG. 8 respectively show the electrical diagrams of Embodiment 1 to Embodiment 5 at different ammonia concentrations.

As shown in FIG. 4, when the ammonia concentration was 10 ppm, the measured current change difference was larger, and the recovery time (i.e., the time for the current to return to its original value after starting to introduce the gas) was 500 seconds. As the concentration of ammonia decreases, the current change difference becomes smaller. When the ammonia concentration was 3 ppm, the current had no significant change, indicating that when the introducing time was 1 second, the minimum sensing concentration of ammonia by the gas sensor of Embodiment 1 was 3 ppm.

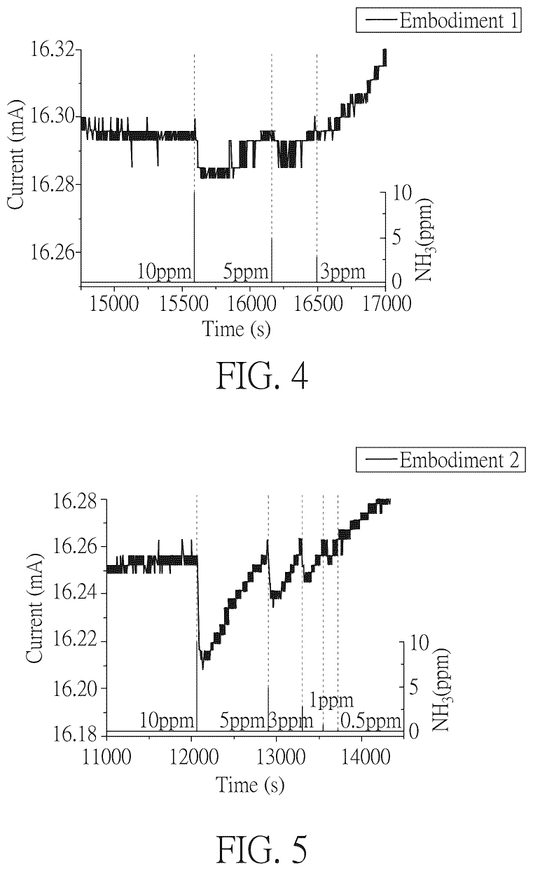

As shown in FIG. 5, compared with FIG. 4, when the introducing time was extended to 5 seconds, with the same ammonia concentration of 10 ppm, the response of the gas sensor of Embodiment 2 (i.e. the current value in the air minus the current value in the ammonia) was significantly higher than the response of the gas sensor of Embodiment 1. Furthermore, the gas sensor of Embodiment 2 also had a longer recovery time of 900 seconds. In addition, while reducing the ammonia concentration, it can be seen that the minimum sensing concentration of ammonia by the gas sensor of Embodiment 2 was 0.5 ppm.

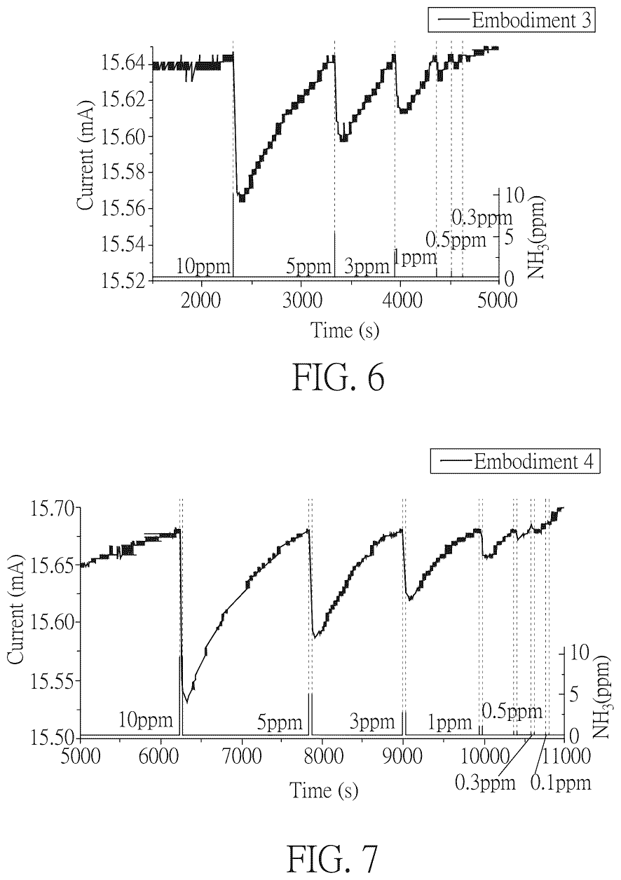

As shown in FIG. 6 to FIG. 8, the introducing time of ammonia was extended to 10 seconds, 30 seconds and 60 seconds respectively. It can be seen that when the ammonia concentration was 10 ppm, the corresponding recovery times were 1000 seconds, 1500 seconds, and cannot return to the initial value beyond 1500 seconds. These results show that the response also increased with the introducing time increased. In addition, the minimum sensing concentration of the gas sensor of Embodiment 3 for ammonia was 0.3 ppm, and the minimum sensing concentrations of the gas sensors of Embodiment 4 and Embodiment 5 for ammonia were 0.1 ppm. These results indicate that the gas sensor of the present embodiment can reach ppb level gas sensing concentration for ammonia.

Relationship between Introducing Time of Gas Sensor and Response

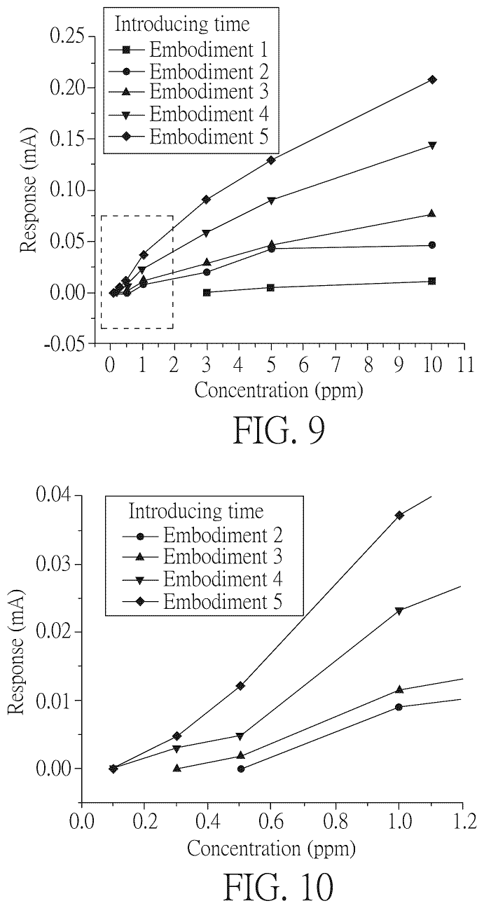

FIG. 9 shows the relationship between different ammonia concentrations and responses from Embodiment 1 to Embodiment 5.

FIG. 10 is a partial enlarged view of the dotted line in FIG. 9.

The result of FIG. 9 is compiled from the results of FIG. 4 to FIG. 8, which plots the changes in ammonia concentration and response at different introducing times. As shown in FIG. 9, when the ammonia concentration is greater than 3 ppm, Embodiment 1 to Embodiment 5 all show a trend that the longer the introducing time, the larger the response. However, when the introducing time of the gas to be measured is greater than 60 seconds and continues to extend, the amplitude of the current decrease will slowly slow down, and the response will reach saturation.

In addition, as shown in FIG. 10 which is a partial enlarged view of the dotted line in FIG. 9, it can be clearly seen that when the ammonia concentration is less than 1 ppm, the same trend is maintained (that is, the longer the introducing time, the greater the response). At the same time, when the ammonia concentration is smaller, the response difference caused by different introducing times is also smaller.

### Test Results of Other Gases

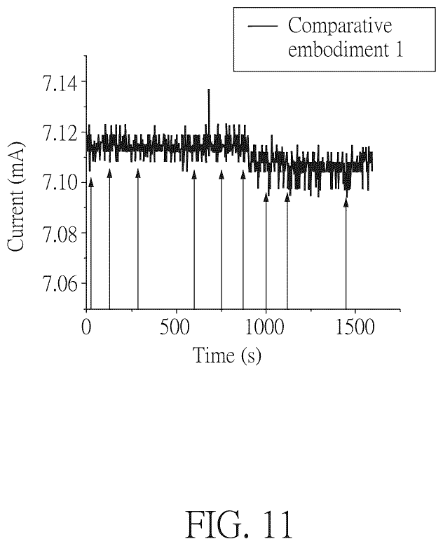

FIG. 11 shows the electrical diagram of Comparative embodiment 1.

As shown in FIG. 11, the arrow in the figure represents the time point when 20 ppm nitrogen dioxide was introduced and lasted for 20 seconds. It can be seen that the current of the gas sensor of the present invention does not change correspondingly and consistently with the introduction of nitrogen dioxide, indicating that nitrogen dioxide cannot cause electrical changes in the gas sensor of the present invention.

In addition, Comparative embodiment 2 to Comparative embodiment 7 also have similar situations to Comparative embodiment 1. That is, the introduced gas to be measured in Comparative Embodiment 2 to Comparative Embodiment 7 has no obvious relationship with the current, indicating that the gas sensor of the present invention has excellent selectivity for ammonia. The gas to be measured in Comparative Embodiment 2 to Comparative Embodiment 7 cannot cause electrical changes in the gas sensor, indicating that the gas sensor of the present invention is not easily interfered by other gases.

In conclusion, in the gas sensor and the gas sensing method using the same of the present invention, the gas sensing layer including the GaN nanowires and the silicon nanowires grown thereon is used to sense gas. For ammonia, the gas sensor of the present invention has significantly improved gas sensing efficiency, can reach ppb level gas sensing concentration at room temperature, or has excellent gas selectivity, which can reduce interference from other gases that affect gas sensing results.

The above effect is because the sensing mechanism of the gas sensor of the present invention can be divided into two parts: in the air environment and in the ammonia environment. Both the GaN nanowires and the silicon nanowires included in the gas sensing layer will participate in the reaction. Therefore, when the gas sensor is placed in the air, the water and oxygen molecules in the air will adsorb to the surface of the silicon nanowires and the electrons on the surface are removed, leaving holes. As the number of water and oxygen molecules adsorbed increases, its adsorption rate and desorption rate will reach equilibrium, leaving a thicker hole accumulation layer on the surface of the silicon nanowires, while reducing the scope of the depletion region of the silicon nanowires. A thicker hole accumulation layer and a smaller depletion region will cause the overall resistance of the silicon nanowires to decrease, so the measured current value will be larger. When ammonia enters the container, ammonia will react with the adsorbed water and oxygen molecules and take them away from the surface of the gas sensing layer. At the same time, the electrons originally removed by water and oxygen molecules will also be released back into the gas sensing layer as they are taken away by ammonia, causing the holes to recombine with the electrons, reducing the thickness of the hole accumulation layer, and increasing the scope of the depletion region. The reduced thickness of the hole accumulation layer and the larger depletion region will increase the resistance of the silicon nanowires, so the measured current will decrease.

In addition, because the length of the silicon nanowires in the gas sensor of the present invention is smaller than the depletion region and the Debye length, it can participate in the entire reaction and increase the overall contact area, further improving the gas sensing efficiency. In addition, the branching structure can effectively block gas molecules, so a lower sensing limit can be obtained. Furthermore, because the entire reaction mechanism is physical and the adsorption and desorption of gas molecules are reversible, the gas sensor of the present invention also has excellent repeatability and durability.

Although the present disclosure has been explained in relation to its embodiment, it is to be understood that many other possible modifications and variations can be made without departing from the spirit and scope of the disclosure as hereinafter claimed.

1. A gas sensor, comprising:
a substrate provided with a gas sensing layer, wherein the gas sensing layer comprises a GaN nanowire and a plurality of silicon nanowires grown on the GaN nanowire;
a first electrode disposed on the substrate; and
a second electrode connected to the gas sensing layer.

2. The gas sensor of claim 1, wherein a diameter of the GaN nanowire ranges from 50 nm to 500 nm.

3. The gas sensor of claim 1, wherein a length of the GaN nanowire ranges from 0.5 μm to 10 μm.

4. The gas sensor of claim 1, wherein one end of the plurality of silicon nanowires away from the GaN nanowire is respectively provided with a catalyst particle, and a material of the catalyst particle is selected from the group consisting of Au, Ag, Ni and an alloy thereof.

5. The gas sensor of claim 1, wherein the second electrode is a grid electrode.

6. The gas sensor of claim 1, wherein the gas sensor is used to sense ammonia.

7. The gas sensor of claim 1, wherein the GaN nanowire and the plurality of silicon nanowires form a branching structure.

8. A gas sensing method, comprising the following steps:
providing a gas sensor, wherein the gas sensor comprises: a substrate provided with a gas sensing layer, wherein the gas sensing layer comprises a GaN nanowire and a plurality of silicon nanowires grown on the GaN nanowire;
a first electrode disposed on the substrate; and a second electrode connected to the gas sensing layer;
placing the gas sensor in a container;
introducing a gas to be measured at a predetermined flow rate for a period of time, and measuring a current change difference of the gas sensor; and
converting the current change difference to obtain a concentration of the gas to be measured.

9. The gas sensing method of claim 8, wherein the predetermined flow rate ranges from 100 mL/min to 1000 mL/min.

10. The gas sensing method of claim 8, wherein the gas to be measured is ammonia.

11. The gas sensing method of claim 8, wherein a diameter of the GaN nanowire ranges from 50 nm to 500 nm.

12. The gas sensing method of claim 8, wherein a length of the GaN nanowire ranges from 0.5 μm to 10 μm.

13. The gas sensing method of claim 8, wherein one end of the plurality of silicon nanowires away from the GaN nanowire is respectively provided with a catalyst particle, and a material of the catalyst particle is selected from the group consisting of Au, Ag, Ni and an alloy thereof.

14. The gas sensing method of claim 8, wherein the second electrode is a grid electrode.

15. The gas sensing method of claim 8, wherein the GaN nanowire and the plurality of silicon nanowires form a branching structure.
